SEQUENCE LISTING (1 sequence)

SEQ ID NO: 1 - DNA, 571 bp, Artificial Sequence
  antisense oligonucleotide of calpain protein
cgttcagccactccatcatgctgagctccacgcttcctgacttatgaaatctcacagtcccctcctgccagctgttcaaacagttttctgaactgatcgtccacatcgtcttcatcgatctcaatcgtctccactctgctctcgacagggtcgtccatctcttgaaagtcagcctgtttctcggagaagacgcggacgcagaagtctccgttcttgtccggttcgaaggtagacgggacgatcaggtattctccgggcggcagacagaagcggctgcacacctccctcgggtcgatgaaagtctcggagcgagcggctgaggcatgacgcaggaagtagttcttattcaggtggacgttcctctgatcgcggaactcatcaggaacctggtagatggcgaagccgaccgtgtgcatgtcctcccccagcttccgggcgcggcgccggttcttctggatcagaccgaccacgaagctgccgccgagctcgcgtcggccgggtcatcgtcttcctcctccagacggatcacaaactgagggttggtccagaacgtgtccgggtagttcctgca

(19) United States
(12) Patent Application Publication
(10) Pub. No.: US 2004/0096869 A1
(43) Pub. Date: May 20, 2004

(54) Antisense calpain nucleotides and uses thereof

(76) Inventors: Daniel Martineau, Saint-Hyacinthe (CA); Zhiying Zhang, Brossard (CA)

Correspondence Address: OGILVY RENAULT, 1981 MCGILL COLLEGE AVENUE, SUITE 1600, MONTREAL, QC, H3A2Y3, CA

(21) Appl. No.: 10/472,496

(22) PCT Filed: Apr. 4, 2002
(86) PCT No.: PCT/CA02/00472

(51) Int. Cl. C12Q 1/68; C07H 21/04; A61K 48/00

(52) U.S. Cl. 435/6; 514/44; 536/23.2

(57) ABSTRACT

The present invention relates to the protection of cells against cell damage and cell death which characterize stroke and myocardial infarction. The invention relates to antisense nucleotides, or nucleotides derived thereof useful for the prevention of cell death and cell damage.

### BACKGROUND OF THE INVENTION

(a) Field of the Invention

The invention relates to the protection of cells against cell damage and cell death which characterize stroke and myocardial infarction. The invention relates to antisense nucleotides, or nucleotides derived thereof useful for the prevention of cell death and cell damage.

(b) Description of Prior Art

Stroke occurs when the blood supply of a brain region is suddenly impaired. Hypoxic neurones release high levels of glutamate at the synaptic junctions. At the postsynaptic membranes, abundant glutamate overactivates the ionotropic excitatory amino acids receptors (EAA) of which the N-methyl-D-aspartate (NMDA), AMPA and kainate receptors. This overactivation, termed excitoxicity, triggers a first influx of calcium ions into neurones whose blood supply was not impaired in the first place. High intracellular calcium concentrations activate calpain and a number of other Ca++-dependent systems such as calmodulin, protein kinase C (PKC), and phospholipase A2. The irreversible activation of PKC leads to aberrant phosphorylation of cellular proteins. Activated calpain is believed to play a major role in neuronal death because it is an efficient protease that causes severe damages to the neuronal cytoskeleton and cytoplasmic membrane. Damaged membranes become permeable to ions, which triggers a second influx of Ca++, and to macromolecules, which ultimately leads to neuronal death.

Currently, the major and most efficient treatment for stroke consists in re-establishing blood flow to the ischemic brain by dissolving the obstructing thrombus and/or preventing thrombosis progression. To achieve this, tissue plasminogen activators or streptokinase are used to promote fibrinolysis, an approach whose inherent risk is the production of intracranial hemorrhages (Muir et al. (1997) Clinical Pharmacology of Cerebral Ischemia, G. J. Ter Horst and J. Korf (eds.), Humana Press, Totowa, N.J. p. 43-66).

Thus far, no treatment has been efficient at protecting neurones from ischemia-induced damage in humans. For instance, calcium ions enter cells through different types of calcium channels, while calcium antagonist drugs are specific with regards with the type of calcium channels they target. Other factors are side effects; antagonists to EAA receptors have been tested to prevent the excitatory cascade. Unfortunately, most of these antagonists have psychotomimetic side-effects (Bartus et al. (1995) Neurological Research, 17:249-258).

Since calpain is a final common pathway in cellular death, the inhibition of calpain by chemical inhibitors provides protection against the activation of a wide array of glutamate receptor subclasses and ion channels. In addition, since proteolysis of cellular proteins by calpain is a late event in the cascade leading to neuronal death, downregulation of calpain will extend the therapeutic window which is currently very narrow.

In contrast with other therapeutic approaches used for brain ischemia, targeting calpain is not likely to trigger acute side effects since calpain is in a latent form in resting cells. (It is activated when cells are overloaded with calcium, that is, in disease) (Wang et al. (1994) Trends in Pharmacological Sciences, 15:412-419). In laboratory animals, chemical calpain inhibitors have successfully reduced the size of cerebral infarcts induced by experimental ischemia. However, major drawbacks of these inhibitors are that they are not specific; they also inhibit a wide range of cellular proteases from different classes. In addition, they poorly penetrate neuronal membranes (Yang et al. (1997) Journal of Neurotrauma, 14:281-297). Antisense therapy may overcome these shortcomings.

Considering the variety of pathways leading to neuronal death in stroke, researchers have concluded that combination therapy is necessary for the efficient treatment of stroke (Wahlgren, N. G. (1997) Neuroprotective agents and cerebral ischaemia, A. R. Green and A. J. Cross (eds.), Academic Press, New York, P. 337-363). Accordingly, calpain-inhibiting drugs might be synergetic with other approaches aiming at re-establishing blood flow or/and at protecting neurones since a final common step in cell death is targeted.

In addition to stroke and myocardial infarction, calpain is also involved in mediating cellular damage in Alzheimer's disease, in brain and spinal cord injury, in muscular dystrophy, in restenosis after coronary angioplasty and in rheumatoid arthritis. It has been shown that chemical calpain inhibitors also protect hypoxic myocytes from damages and death induced by ischemia ((Wang et al. (1994) Trends in Pharmacological Sciences, 15:412-419) and (Yoshida et al. (1995) Circulation Research, 77:603-610)).

Traditional therapeutic methods are generally based on protein interactions. This results in a lack of specificity and/or in frequent side effects. In contrast, antisense technology is highly specific and thus should not have side effects since it uses poly- or oligonucleotides whose sequence is complementary to, and thus highly specific for the target mRNA. Consequently, protein synthesis is prevented, and expression of the target gene is inhibited.

Antisense therapy uses two major approaches. The first one is the administration of short synthetic antisense molecules (oligonucleotides) . The second is the local delivery of vectors with the aim of achieving stable, intracellular expression of much longer antisense RNA (polynucleotides). In both approaches, the selection of an appropriate target sequence is very arduous since, for unclear reasons, only few antisense sequences bind efficiently in vivo to the target RNA. As a result, in order to design a single efficient antisense oligonucleotide, 30 to 40 molecules have to be screened to find an oligonucleotide that efficiently downregulates gene expression.

It would therefore be advantageous to be provided with an antisense nucleotide sequence which could bind and efficiently trigger the destruction of the target mRNA that efficiently downregulates calpain expression and thereby alleviates the effect of calpain activity.

### SUMMARY OF THE INVENTION

It is an aim of the present invention to provide an antisense calpain nucleotide sequence capable of hybridizing with a mRNA encoding for a protease of the calpain family and thus downregulating the expression of the protease of the calpain family.

It is an aim of the present invention to provide an antisense calpain nucleotide sequence capable of hybridizing with a mRNA encoding for a protease of the calpain family and thus capable of protecting cells from damage and death.

It is also an aim of the present invention to provide the use of the antisense calpain nucleotide sequence of the present invention for the treatment or prevention of ischemic syndromes caused by insufficient cerebral circulation.

In accordance with the present invention, there is provided an antisense calpain nucleotide sequence capable of hybridizing with a mRNA encoding for a protease of the calpain family and thus downregulating the expression of the protease of the calpain family.

In accordance with the present invention, there is provided an antisense calpain nucleotide sequence capable of hybridizing with a mRNA encoding for a protease of the calpain family and thus capable of protecting cells from damage and death.

In accordance with the present invention, there is provided an antisense nucleotide capable of hybridizing with a mRNA encoding for a protease of the calpain family, capable of triggering their destruction and thus capable of protecting cells from damage and death.

In accordance with the present invention, there is provided an antisense calpain nucleotide sequence capable of hybridizing to the calpain mRNA region corresponding to the 3′ half of Domain III of the calpain large subunit.

### DETAILED DESCRIPTION OF THE INVENTION

In a preferred embodiment, there is provided an antisense calpain sequence (571 base pairs) as follows:

| | |
|---|---|
| 1. | CGTTCAGCCA CTCCATCATG CTGAGCTCCA CGCTTCCTGA CTTATGAAAT |
| 51. | CTCACAGTCC CCTCCTGCCA GCTGTTCAAA CAGTTITCTG AACTGATCGT |
| 101. | CCACATCGTC TTCATCGATC TCAATCGTCT CCACTCTGCT CTCGACAGGG |
| 151. | TCGTCCATCT CTTGAAAGTC AGCCTGTTTC TCGGAGAAGA CGCGGACGCA |
| 201. | GAAGTCTCCG TTCTTGTCCG GTTCGAAGGT AGACGGGACG ATCAGGTATT |
| 251. | CTCCGGGCGG CAGACAGAAG CGGCTGCACA CCTCCCTCGG GTCGATGAAA |
| 301. | GTCTCGGAGC GAGCGGCTGA GGCATGACGC AGGAAGTAGT TCTTATTCAG |
| 351. | GTGGACGTTC CTCTGATCGd GGAACTCATC AGGAACCTGG TAGATGGCGA |
| 401. | AGCCGACCGT GTGCATGTCC TCCCCCAGCT TCCGGGCGCG GCGCCGGTTC |
| 451. | TTCTGGATCA GACCGACCAC GAAGCTGCCG CCGAGCTCGC GTCGGCCGGG |
| 501. | TCATCGTCTT CCTCCTCCAG ACGGATCACA AACTGAGGGT TGGTCCAGAA |
| 551. | CGTGTCCGGG TAGTTCCTGC A; |

or an analog thereof.

In accordance with the present invention, there is provided a 571-bp antisense calpain nucleotide sequence as described above or an analog thereof wherein said antisense sequence or analog thereof is capable of hybridizing with a portion of a target mRNA encoding for a protein of the calpain family wherein said hybridization downregulates the expression of a protein of the calpain family.

In accordance with the present invention, there is provided a method for downregulation of the expression of proteins of the calpain family comprising the step of administering an antisense calpain nucleotide sequence having a nucleotide sequence capable of hybridizing with mRNAs encoding for the various members of the calpain protease family, capable of triggering their destruction and thus capable of protecting cells from damage and death.

The antisense calpain nucleotide sequence of the present invention is useful for preventing pathological conditions characterized by cell death and degeneration to which calpain activation contributes. These conditions are: cerebral ischemia (including ischemia caused by cerebral vasospasm induced by subarachnoid hemorrhages), traumatic brain injury, spinal cord injury, multiple sclerosis, myocardial infarction, restenosis after coronary angioplasty, muscular dystrophy, cataract, thrombotic platelet aggregation and rheumatoid arthritis.

In a further preferred embodiment, there is provided antisense calpain nucleotide sequence capable of hybridization with at least a portion of a target mRNA of mammalian m-calpain wherein said hybridization downregulates the expression of mammalian m-calpain.

In a further preferred embodiment, there is provided antisense calpain nucleotide sequence capable of hybridization with at least a portion of a target mRNA of mu-calpain wherein said hybridization downregulates the expression of mu-calpain.

The antisense calpain nucleotide sequence of the present invention can also be used as a probe for detecting or isolating sequences encoding for proteins of the calpain family.

The antisense calpain nucleotide sequence of the present invention can be cloned or replicated by methods well known in the art such as PCR (Polymerase Chain Reaction). All nucleotides obtained by such methods are encompassed by the present invention.

Pharmaceutically acceptable liposomal compositions comprising the antisense nucleotide of the present invention in combination with a pharmaceutically acceptable carrier are included in the scope of the present invention and can be prepared by methods well known in the art.

The nucleotide sequence of the present invention can be administered in combination with a second therapeutic agent such as thrombolytic agents.

The nucleotide sequence of the present invention inserted in a viral or non-viral vector can be administered alone or in combination with a second therapeutic agent such as thrombolytic agents.

Thrombolytic agents include but are not limited to tissue plasminogen activator and streptokinase.

All the methods used for the delivery of DNA into nervous cells, both in vivo and in vitro, are included in the scope of the present invention. These methods include viral vectors derived from retroviruses, Herpes viruses, adenoviruses, and non viral methods and/or vectors (chemical, physical, receptor-mediated, and cationic liposome-mediated transfection). These methods are well-known in the art and are reviewed in Yang et al. ((1997) Journal of Neurotrauma, 14:281-297). The use of cationic lipids to enhance the biological activity of antisense nucleotides is reviewed by Bennett et al. ((1993) Journal of Liposome Research; 3:85-102).

In a further embodiment of the present invention, it is possible to use cationic liposomes to increase transfection efficiency. These cationic liposomes increase transfection efficiency both in vivo and in vitro when they are used as carriers. They have low toxicity, are non immunogenic, and are easy to prepare and use. Liposome-mediated transfection is also efficient in non-dividing cells.

The antisense calpain nucleotide sequence for the present invention can be administered by direct intracerebral, intraarterial (carotid), intravenous, intraocular, intraarticular, oral, enteric and/or rectal, and vaginal routes.

By the term “or an analog thereof” is meant a nucleotide sequence which shows at least 65% homology with the 571-bp antisense of calpain, preferably at least 75% homology with the 571-bp antisense of calpain, and, more preferably, at least 85% homology with the 571-bp antisense of calpain.

By the term “hybridization” is meant a process by which a strand of nucleic acid joins with a complementary strand through base pairing. In order for the pair of nucleic acid strands to be hybridized, there has to be at least 65% of homogeneity between the strands, preferably at least 75% and, more preferably, at least 85%.

By the terms “target m-RNA” and “mRNA” are meant an m-RNA or a pre-m-RNA encoding for a protease of the calpain family.

By the term “protease of the calpain family” is meant a protein having a biological activity similar to the biological activity of mammalian m-calpain and mu-calpain.

Members of the calpain family modulate precisely and irreversibly the activities and functions of other cellular proteins. Their activity is regulated by intracellular calcium concentration. The two major isoforms, mu-calpain and milli-calpain, are ubiquitous, i.e. they are present in most tissues and their activation requires micro and millimolar Ca++ concentrations, respectively. Other members of the calpain family are tissue-specific. nCL-1 calpain (p94) is present in skeletal muscle; nCL-2 is present in stomach; nCL-2 and nCL2′ are present in skeletal muscle and stomach, respectively, and nCL-4 is found in stomach and intestines. Both ubiquitous and tissue-specific calpains are heterodimers consisting of a distinct large (80 kDA) and a common small subunit (29 kDa). The large subunit is divided in 4 domains. Domain II is the catalytic domain homologous to other cysteine proteases. Domain IV, through which the two subunits are associated, has four EF-hand Ca++ binding sites. Domains I and III have no homology to other proteins.

The small subunit is composed of two domains. Domain V (N-terminus) is hydrophobic and Domain VI (C-terminus), homologous to Domain IV of the large subunit, also binds Ca++ (Sorimachi et al. (1997) Biochemical Journal, 328:721-732).

By the term “downregulating” is meant a diminution of the production of a protein of the calpain family in comparison to a standard.

The walleye dermal sarcoma virus (WDSV), a retrovirus, is the etiological agent of a tumor endemic in populations of walleye fish throughout North America. In some lakes, the tumor seasonally affects one fish out of four. It was reported that WDS is the only retrovirus-induced sarcoma that occurs in nature with a significant prevalence. Although this tumor appears histologically malignant, it neither invades nor metastasizes in the natural disease. Tumor development occurs in the fall and continues throughout winter. In the spring, tumors regress on most affected fish in the course of several weeks, during spawning. Macroscopically, regression is manifested by tumors falling off, and affected fish are not affected otherwise. Microscopically, tumor regression is characterized by coagulation necrosis of tumors, suggestive of ischemia.

The present invention will be more readily understood by referring to the following examples which are given to illustrate the invention rather than to limit its scope.

### EXAMPLE I

### Production of Antisense Calpain Nucleotide

The antisense calpain was obtained by RT-PCR from tumor tissue, by methods described in Ausubel et al. ((1997), Current protocols in molecular biology, Ausubel, F. M. et al. (eds.), John Wiley & Sons, Inc., p. 15.0.1-15.8.8). We have amplified, cloned and sequenced a viral cDNA containing a 571-bp long nucleotide sequence with high homology (Table 1) to the negative strand of the human m-calpain large subunit (70.4%), to the negative strand of the human mu-calpain large subunit (65.1%), and to the negative strand of the human muscle-specific calpain large subunit (68.5%). This antisense calpain-like sequence is flanked by the viral long terminal repeats (LTR) of WDSV. By RT-PCR followed by Southern blot, we have shown the presence of this transcript in 18% of tumors (3 tumors out of 17) using primers specific for the viral LTR and for the antisense calpain-like transcript.

The viral antisense transcript was sequenced. The region where it is predicted to bind to the calpain sequence was identified. The cDNA was cloned and placed under the control of a strong eukaryote promoter (CMV). The corresponding region of the walleye fish cellular cDNA calpain gene was amplified, cloned and sequenced using RT-PCR with primers deduced from the viral calpain transcript containing the antisense calpain sequence. A 954-bp nucleotide sequence was amplified. Within this sequence, a 510-bp nucleotide region was 99.4% identical to the negative strand of the viral antisense sequence, i.e. 3 nucleotides were different out of 510.

Table 1 shows the region of homologies between the WDSV antisense calpain sequence and the human calpain protein.

TABLE 1
Region of homologies between the WDSV antisense
calpain sequence and human members of the calpain
family

| Our | Human milli | | Human mRNA for |
|---|---|---|---|
| inven- | cANP large | | skeletal muscle- |
| tion | subunit | Human mu cANP large | specific calpain |
| (bp- | M23254 | subunit X04366 | X85030 |
| number) | (bp-number) | (bp-number) | (bp-number) |
| 1-571 | Domain III | Domain III | Domain III |
| | boundaries: | boundaries: | boundaries: |
| | 952-1668 | 1125-1850 | 1203-1959 |
| | Homology: | Homology: | Homology: |
| | 1250-1776 | 1293-1825 | 1339-1734 |
| | (70.4% identity | (65.1% identity | (68.5% identity |
| | in 527 bp over- | in 533 bp overlap) | in 403 bp overlap) |
| | lap) | | |

### EXAMPLE II

### Biological Activities of the Antisense Compound of the Invention

General objective. The fish antisense calpain sequence protects cells from calpain-mediated damage. Sub-objective. To demonstrate that the fish antisense calpain transcript inhibits calpain expression

Outline: transiently and stably transfect various cell lines with the fish antisense calpain and quantitate calpain mRNA and proteins.

Transient transfection. Five human cell lines derived from various tissues (fibroblast, cervical epithelium, lung alveolar epithelium, neuroblastoma, liver) are transiently transfected with the fish antisense placed under the control of a strong constitutive promoter (CMV promoter), active in most cell lines.

Controls: to confirm that the effect is due to the antisense sequence (and not singly to the transfected sequence, independently of its orientation), a first control is used: it is an insert having the same size as the fish antisense sequence, with no apparent open reading frame, and whose base composition is similar to the fish antisense. A second control is the fish antisense inserted in the “sense” orientation.

Increasing amounts of plasmids are used to show a dose-response. At different times after transfection (6,12,18 and 24 hours), total RNA is isolated and analyzed by Northern blot using an oligonucleotide probe specific for the human calpain large subunit. A probe specific for glycerol-3-phosphate dehydrogenase (G3PDH) is used for normalization of calpain mRNA levels. RNA levels are measured by densitometry.

Proteins are extracted at the same time points as RNA. Calpain activity is determined directly using the casein zymography assay (Raser et al. (1995) Archives of Biochemistry & Biophysics, 319:211-216). Calpain protein levels are determined by Western blot using antibodies against m-calpain and mu-calpain. Immunoreactive proteins are quantitated by densitometry. Spectrin is a substrate for calpain-induced proteolysis. To determine whether calpain expression and activation are decreased by the expression of the antisense transcript, both in vivo and in vitro, we will determine the extent of alpha spectrin degradation by Western blot, and the levels of m-calpain and mu calpain by Western and Northern blots.

To find whether a specific region of the construct is involved in calpain expression, constructs having 5′ sequential, progressive deletions are generated, using exonuclease III. These constructs are transfected in cells submitted to hypoxia, or treated with ionophores. When the most efficient antisense region is deleted from the antisense construct, there is no inhibition of calpain activity. Oligonucleotides corresponding to the identified regions are synthesized, and used to treat untransformed cells challenged by hypoxia and ionophores. Controls are scrambled oligonucleotides with the same base composition as the oligonucleotide under study. If the correct target region has been identified, there should be inhibition of calpain activity.

In vivo, the Expression of the Antisense Calpain-like Molecule Confers Resistance to Calpain-induced Cell Death

To determine whether the antisense compound of the invention also prevents cell death in vivo, in rats subjected to experimental cerebral infarcts, the following procedures are followed.

Naked DNA construct carried by cationic liposomes is injected directly in the brain. The choice of this route of administration is based on previous work by Weiss et al. (“Antisense” meeting, (1997) Cambridge, Mass. 35) and Life Sciences (1997), 60:433-455). Expression of the construct is determined following intracerebral injection in normal brain by in situ hybridization using a sense specific riboprobe.

To generate cerebral infarcts, a variation of the craniotomy middle cerebral artery (MCA) occlusion method is used where the left MCA is ligated, while the common carotid arteries are bilaterally and temporarily occluded. Three groups of animals are used: the first group receives an injection of the antisense calpain-like construct 6 hours prior to experimental infarction. The injection is in the left MCA that will be later occluded. The second group receives the treatment one hour after infarction and the third group receives the treatment three hours after infarction. This particular schedule is based on a study that showed that calpain inhibitors injected in the brain significantly decrease infarct volume. Twenty-one hours after induction of focal ischemia, rats are sacrificed, their brain processed as described in Bartus et al. ((1994) Journal of Cerebral Blood Flow & Metabolism, 14:537-544). Briefly, brains will be sectioned and stained with 2,3,5-triphenyltetrazolium chloride (TTC) to stain the infarct areas. Computer digitization of each section, and subsequent image analysis is undertaken, also as described in Bartus et al. ((1994) Journal of Cerebral Blood Flow & Metabolism, 14:537-544).

Different combinations of oligonucleotides and vector are tested based on the following rationale. After oligonucleotides penetrate injured cells, they bind mRNA in minutes while the expression of antisense calpain from vectors might take hours.

The antisense of the present invention can be delivered to the nervous system by methods which are well-known in the art. There are several reports of successful expression of therapeutic naked DNA vectors injected directly into the brain. For instance, the lipofectin-mediated tyrosine hydroxylase gene has been transfected in animal models of Parkinson's disease by direct injection in rat striatum (Cao et al. (1997) Human Gene Therapy, 6:1497-1501). The cholecystokinin (CCK) gene was injected in the cerebral ventricles of rats with audiogenic seizures. After the treatment, suppression of audiogenic seizures was accompanied by increased CCK mRNA in the brain (Zhang et al. (1997) Neuroscience, 77:15-22). It was also demonstrated that intrastriatal injection of naked antisense D2 dopamine receptor oligonucleotides inhibits D2 dopamine receptor-mediated behavior in mouse. Recently, an expression vector, administered in a single intrastriatal injection to generate an antisense RNA to D2 dopamine receptor, also selectively reduced the levels of D2 dopamine receptors, and caused selective, long-term (one month) inhibition of pathological behaviors triggered by D2 dopamine agonists ((Hadjiconstantinou et al. (1996) Neuroscience Letters, 217:105-108), (Weiss, B. (1997) “Antisense” meeting, Cambridge, Mass. 35) and (Weiss et al. (1997) Life Sciences 60:433-455)).

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features herein before set forth, and as follows in the scope of the appended claims.

What is claimed is:

1. An antisense calpain nucleotide sequence capable of hybridizing with a mRNA encoding for a protease of the calpain family and thus downregulating the expression of the protease of the calpain family.

2. An antisense calpain nucleotide sequence capable of hybridizing with a mRNA encoding for a protease of the calpain family and thus capable of protecting cells from damage and death.

3. An antisense calpain nucleotide sequence capable of hybridizing with a mRNA encoding for a protease of the calpain family, capable of triggering their destruction and thus capable of protecting cells from damage and death.

4. An antisense calpain sequence capable of hybridizing with the calpain mRNA region corresponding to the 3′ half of Domain III of the calpain large subunit.

5. An antisense calpain oligonucleotide having nucleotide sequences consisting of:

| | |
|---|---|
| 1. | CGTTCAGCCA CTCCATCATG CTGAGCTCCA CGCTTCCTGA CTTATGAAAT |
| 51. | CTCACAGTCC CCTCCTGCCA GCTGTTCAAA CAGTTTTCTG AACTGATCGT |
| 101. | CCACATCGTC TTCATCGATC TCAATCGTCT CCACTCTGCT CTCGACAGGG |
| 151. | TCGTCCATCT CTTGAAAGTC AGCCTGTTTC TCGGAGAAGA CGCGGACGCA |
| 201. | GAAGTCTCCG TTCTTGTCCG GTTCGAAGGT AGACGGGAcG ATCAGGTATT |
| 251. | CTCCGGGCGG CAGACAGAAG CGGCTGCACA CCTCCCTCGG GTCGATGAAA |
| 301. | GTCTCGGAGC GAGCGGCTGA GGCATGACGC AGGAAGTAGT TCTTATTCAG |
| 351. | GTGGACGTTC CTCTGATCGC GGAACTCATC AGGAACCTGG TAGATGGCGA |
| 401. | AGCCGACCGT GTGCATGTCC TCCCCCAGCT TCCGGGCGCG GCGCCGGTTC |
| 451. | TTCTGGATCA GACCGACCAC GAAGCTGCCG CCGAGCTCGC GTCGGCCGGG |
| 501. | TCATCGTCTT CCTCCTCCAG ACGGATCACA AACTGAGGGT TGGTCCAGAA |
| 551. | CGTGTCCGGG TAGTTCCTGC A; |

or an analog thereof.

6. The use of the antisense calpain as described in anyone of claims 1 to 5 for the treatment or prevention of ischemic syndromes caused by insufficient cerebral circulation.

7. The use of the antisense calpain sequence as described in any one of claims 1 to 5 for preventing cell death and degeneration associated with diseases and conditions such as Parkinson's disease, Alzheimer's disease, multiple sclerosis, myocardial infarction, muscular dystrophy, cataract, thromboic platelet aggregation, restenosis after coronary angioplasty, and rheumatoid arthritis.
